(19) United States
(12) Patent Application Publication
(10) Pub. No.: US 2003/0031676 A1
(43) Pub. Date: Feb. 13, 2003

(54) Conjugate compounds for treating atheroma and other diseases

(75) Inventors: Jonathan L. Sessler, Austin, TX (US); Darren Magda, Cupertino, CA (US)

Correspondence Address: VINIT G. KATHARDEKAR, PHARMACYCLICS, INC., 995 E. ARQUES AVENUE, SUNNYVALE, CA, 94085, US

(73) Assignee: Pharmacyclics, Inc.

(21) Appl. No.: 10/160,205

(22) Filed: May 30, 2002

Related U.S. Application Data
(63) Continuation of application No. PCT/US00/29524, filed on Oct. 27, 2000.
(60) Provisional application No. 60/304,197, filed on Oct. 29, 1999.

(51) Int. Cl. A61K 38/17; A61K 31/7052; A61K 39/395; C07D 487/22; C07K 14/775; C07K 16/46; A61K 9/127

(52) U.S. Cl. 424/178.1; 514/23; 514/185; 514/250; 514/229.8; 514/12; 514/410; 530/359; 530/391.1; 536/17.4; 540/145; 540/465; 540/472; 540/474; 424/450

(57) ABSTRACT

Disclosed are compounds which are conjugates of (a) a moiety capable of localizing in the cells of a tumor or atheroma and (b) a moiety capable of catalyzing the production of reactive oxygen species from a cellular metabolite. The disclosed compounds which are useful for treating atheroma, tumors and other neoplastic tissue.

### CROSS-REFERENCE TO RELATED APPLICATIONS

This is a continuation-in-part of co-pending U.S. patent application Ser. No. 09/431,298, filed Oct. 29, 1999, converted to provisional U.S. Patent Application Serial No. ______, incorporated herein by reference.

### BACKGROUND OF THE INVENTION

1. Field of the Invention

This invention relates to compounds that are useful for treating atheroma, tumors and other neoplastic tissue. More specifically, this invention provides compounds that are conjugates of (a) a moiety capable of localizing in the cells of a tumor or atheroma and (b) a moiety capable of catalyzing the production of reactive oxygen species from a cellular metabolite.

### PUBLICATIONS CITED BY REFERENCE

Certain publications are cited in this application through the use of the following superscript numbers:

¹ Buettner, et al., Radiation Research, Catalytic Metals, Ascorbate and Free Radicals: Combinations to Avoid, 145:532-541 (1996)

² Isoda, et al., J. Cancer Research, Change in Ascorbate Radical Production in an Irradiated Experimental Tumor with Increased Tumor Size, 56:5741-5744 (1996)

³ Riley, Int. J. Radiat. Biol., Free Radical in biology: oxidative stress and the effects of ionizing radiation, 65(1):27-33 (1994)

⁴ Sessler, et al., J. Phys. Chem. A, One-Electron Reduction and Oxidation Studies of the Radiations Sensitizer Gadolinium (III) Texaphyrin (PCI-120) and Other Water Soluble Metallotexaphyrins, 103: 787-794 (1999)

⁵ Adams, et al., Radiation Res., 67:9-20 (1976)

⁶ Riley, Int. J. Radiat. Biol., Free Radicals in Biology: Oxidative Stress and the Effects of Ionizing Radiation, 65(1):27-33 (1994).

⁷ Volpin, et al., WO97/03666, EP 0 786 253 A1, U.S. Pat. No. 6,004,953, Agent for Suppressing Tumor Growth

⁸ Young, et al., U.S. Pat. No. 5,776,925, Methods for Cancer Chemosensitization, issued Jul. 7, 1998

⁹ Sessler, et al., U.S. Pat. No. 6,072,038, Conjugates of Texaphyrins, issued Jun. 6, 2000

¹⁰ Sessler, et al., U.S. application Ser. No. 09/325,890, filed Jun. 4, 1999, allowed, Texaphyrin Conjugates and Uses Thereof.

¹¹ Young, et al., WO97/46262, Membrane Incorporation of Texaphyrins.

All of the above publications are herein incorporated by reference in their entirety to the same extent as if each individual publication was specifically and individually indicated to be incorporated by reference in its entirety.

2. Background Information

The treatment of solid mammalian tumors with ionizing radiation involves the in situ generation of hydroxyl radicals and other reactive oxygen species which, due to the focusability of the ionizing radiation are primarily located in the tumor, i.e., in tumor cells. These reactive species possess extreme oxidizing properties which oxidize biomolecules in vivo thereby interfering with cellular metabolism.¹ For example, it is reported that ionizing radiation, such as X-rays and y-rays, induces irreversible damage to cellular DNA through production of hydroxyl radicals and other reactive oxygen species in the cell leading to cell death^{2,3} or initiation of the mechanism of apoptosis.⁴

One generally accepted mechanism of the cellular effect of ionizing radiation is initial damage inflicted to the cell's DNA by reactive oxygen species generated by the ionizing radiation. In the presence of molecular oxygen, this damage is largely irreparable. Contrarily, in the absence of molecular oxygen (such as hypoxic cells), cellular antioxidants such as ascorbate, NADH and NADPH can act to repair damage to the tumor DNA.

Tumor treatment via the use of ionizing radiation can be enhanced by increasing the radiosensitivity of the tumor cells. One method suggested for enhancing radiosensitivity has been the external administration of a compound having a high affinity for electrons, which ideally localizes in the tumor. Proposed radiation sensitizers include compounds such as halogenated pyrimidines, nitroimidazoles and gadolinium (III) complexes of the pentadentate macrocycle texaphyrin.⁴ Motexafin gadolinium (a gadolinium (III) texaphyrin complex) is currently in Phase III clinical trials for the treatment of brain metastheses.⁴

The observation that radiation sensitization occurs as a function of redox potential gave rise to the proposal that such compounds function by interception of aqueous electrons, thus preventing their recombination with cytotoxic radicals.⁵ Subsequent evidence showing a lack of radiation sensitization activity for lutetium (III) texaphyrin in animal models notwithstanding the rapidity of reaction between this complex and hydroxyl radicals under pulsed radiolytic conditions and minimal apparent nuclear localization suggest that this proposal might not fully explain the mechanism by which the gadolinium texaphyrins act as radiosenstizers.⁴

Phthalocyanine and naphthalocyanine polydentate ligands of the transition metals cobalt and iron have been described as suppressing the growth of tumor cells when administered in combination with a biogenic reductant such as ascorbic acid.⁷

As disclosed in co-pending application PCT/US00/______ and its counterpart U.S. patent application Ser. No. ______, filed on even date herewith and entitled “METHODS AND COMPOSITIONS FOR TREATING ATHEROMA, TUMORS AND OTHER NEOPLASTIC TISSUE” (Attorney Docket No. 4202.01), which is a continuation-in-part of U.S. patent application Ser. No. 09/430,505, filed Oct. 29, 1999 and converted to provisional U.S. Patent Application Serial No. ______, the disclosure of which is incorporated herein by reference in its entirety, it has now been discovered that the known radiation sensitizer motexafin gadolium acts to catalyze the oxidation of NADH, NADPH, ascorbate and other reducing agents under approximate physiological conditions, to generate reactive oxygen species, such as superoxide and hydrogen peroxide.⁶ Metallotexaphrins are known to localize in atheroma, tumor cells and other neoplastic tissue. The generation of reactive oxygen species in situ facilitates oxidative attack on the tumor or other tissue, leading to targeted oxidative stress and effecting treatment and/or sensitization to radiation where such a reactive oxygen species is generated.

The administration of texaphyrins with other active agents has been described, for example, with chemosensitizers⁹, and various texaphyrin conjugates have also been described, for example, including: with biomolecules,⁹ with chemotherapeutic moieties (e.g., cisplatin)¹⁰ and in texaphyrin-lipophilic molecule-vessicle complexes¹¹.

The above-referenced discovery concerning radiation sensitization has further provided the ability to evaluate the radiation sensitization potential of other known and new compounds, as a function of their ability to catalyze the generation of reactive oxygen species from cellular metabolites under approximate physiologic conditions. While some of the compounds found to have radiation sensitization potential will have the inherent ability to localize in atheroma, tumors or other neoplastic tissues, other such reactive oxygen species catalysts may not be useful for treatment in and of themselves due to an inability to selectively localize. The present invention is addressed to facilitating the selective localization of such reactive oxygen species catalysts to provide a new class therapeutic agents for treating conditions responsive to the induction of targeted oxidative stress.

### SUMMARY OF THE INVENTION

This invention is directed to compounds which localize in tumor or atheroma cells and which catalyze the in situ production of reactive oxygen species from cellular metabolites. Accordingly, when administered to a mammalian host having a tumor or atheroma or other condition responsive to targeted cellular oxidative stress, the compounds of this invention selectively catalyze the production of reactive oxygen species in the targeted cells thereby killing or treating the tumor, atheroma or other condition, or rendering it more susceptible to treatment with ionizing radiation.

In one of its composition aspects, this invention is directed to a compound comprising a conjugate of (a) a moiety capable of localizing in the cells of a tumor or atheroma or other neoplastic tissue, and (b) a moiety capable of catalyzing the production of hydrogen peroxide from a cellular metabolite having a standard biochemical reduction potential more negative than the standard biochemical reduction potential of oxygen/hydrogen peroxide (but, typically, not capable of localizing in the cells of a tumor or atheroma), or a pharmaceutically acceptable salt thereof.

In another of its composition aspects, this invention is directed to a compound of formula I:

A-[-L-X]ₙ I

wherein:

A is a moiety capable of localizing in the cells of a tumor or atheroma;

each X is independently a moiety capable of catalyzing the production of reactive oxygen species from a cellular metabolite having a standard biochemical reduction potential more negative than the standard biochemical reduction potential of oxygen/hydrogen peroxide;

each L is independently a linking group covalently attaching X to A;

n is an integer ranging from 1 to 5;

and pharmaceutically acceptable salts thereof.

In one embodiment, A in formula I is preferably a metallotexaphyrin. In another embodiment, A is a porphyrin, metalloporphyrin, antibody, low density lipoprotein, saccharide, or a lipophilic hydrocarbyl moiety capable of association with a liposome.

Preferably, each X in the compound of formula I is independently selected from the group consisting of alloxan, phenazonium salts, a quinone and derivatives and/or salts thereof.

Each L in the compounds of formula I is preferably independently selected from the group consisting of a covalent bond, an alkylene group and a poly(oxyalkylene) group, optionally including an amidocarboxy or carboxamide functionality.

Preferably, in formula I, n is 1 or 2.

Another embodiment of this invention provides conjugates of (a) a moiety that localizes in the cells of a tumor or atheroma and which is capable of catalyzing the production of reactive oxygen species from a cellular metabolite, and (b) a ligand that binds to NADH or NADPH. In these compounds, binding of the ligand to NADH or NADPH improves the activity of the moiety that catalyzes the production of reactive oxygen species.

Accordingly, in another of its composition aspects, this invention provides compounds of formula II:

B-[-L-Y]ₙ II

wherein

B is a moiety capable of localizing in the cells of a tumor or atheroma and which is capable of catalyzing the production of reactive oxygen species from a cellular metabolite having a standard biochemical reduction potential more negative than the standard biochemical reduction potential of oxygen/hydrogen peroxide;

each Y is independently a ligand capable of binding to NADH or NADPH;

each L is independently a linking group covalently attaching Y to B; and

n is an integer ranging from 1 to 5;

and pharmaceutically acceptable salts thereof.

In one embodiment, B in formula II is preferably a metallotexaphyrin. In another embodiment, B is a porphyrin, metalloporphyrin, antibody, low density lipoprotein, saccharide, or a lipophilic hydrocarbyl moiety capable of association with a liposome.

Preferably, each Y in the compound of formula II is thymine or a derivative thereof.

Each L in the compounds of formula II is preferably independently selected from the group consisting of a covalent bond, an alkylene group and a poly(oxyalkylene) group, optionally including an amidocarboxy or carboxamide functionality.

Preferably, in formula II, n is 1 or 2.

In yet another of its composition aspects, this invention is directed to a pharmaceutical composition comprising a pharmaceutically acceptable carrier and a therapeutically effective amount of a compound comprising a conjugate of (a) a moiety capable of localizing in the cells of a tumor or atheroma or other neoplastic tissue and (b) a moiety capable of catalyzing the production of reactive oxygen species from a cellular metabolite having a standard biochemical reduction potential more negative than the standard biochemical reduction potential of oxygen/hydrogen peroxide, or a pharmaceutically acceptable salt thereof.

This invention is also directed to pharmaceutical compositions comprising a pharmaceutically acceptable carrier and a therapeutically effective amount of a compound a compound of formula I or II.

In another of its aspects, this invention is directed to the use of a compound of this invention, including compounds of formula I and formula II above, in the manufacture of a formulation or medicament for treating an atheroma or tumor or other neoplastic tissue in a mammalian host.

In one of its method aspects, this invention provides a method of treating a mammalian host having a tumor or atheroma or other neoplastic tissue, the method comprising:

(A) administering to a mammalian host having a tumor or atheroma an effective amount of a compound comprising a conjugate of (a) a moiety capable of localizing in the cells of a tumor or atheroma and (b) a moiety capable of catalyzing the production of reactive oxygen species from a cellular metabolite having a standard biochemical reduction potential more negative than the standard biochemical reduction potential of oxygen/hydrogen peroxide, or a pharmaceutically acceptable salt thereof.

This invention is further provides methods of treating a mammalian host having a tumor or atheroma or other neoplastic tissue, the method comprising:

(A) administering to a mammalian host having a tumor or atheroma an effective amount of a compound of formula I or II.

In another embodiment, either the above methods further comprises the step of:

(B) exposing the tumor or atheroma or other neoplastic tissue to ionizing radiation.

### BRIEF DESCRIPTION OF THE DRAWINGS

FIG. 1 illustrates the chemical structure of texaphyrin compounds.

FIG. 2 illustrates the chemical structure of various cellular redox mediators.

FIGS. 3-5 show the preparation of conjugate compounds of this invention.

FIG. 6 shows the binding of a compound of formula II to NADH and NADPH.

### DETAILED DESCRIPTION OF THE INVENTION

This invention is directed to compounds which localize in tumor or atheroma or neoplastic tissue cells and which catalyze the production of reactive oxygen species from cellular metabolites; and to pharmaceutical compositions and methods employing such compounds. When defining the compounds, compositions and methods of this invention, the following terms have the following meanings, unless otherwise indicated.

Definitions

The term “alkyl” refers to a monoradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group as defined above, having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkylene” refers to a diradical of a branched or unbranched saturated hydrocarbon chain, preferably having from 2 to 6 carbon atoms, more preferably 2 to 4 carbon atoms. This term is exemplified by groups such as ethylene (—CH₂CH₂—), the propylene isomers (e.g., —CH₂CH₂CH₂— and —CH(CH₃)CH₂—) and the like.

The term “substituted alkylene” refers to an alkylene group, as defined above, having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Additionally, such substituted alkylene groups include those where 2 substituents on the alkylene group are fused to form one or more cycloalkyl, substituted cycloalkyl, cycloalkenyl, substituted cycloalkenyl, aryl, heterocyclic or heteroaryl groups fused to the alkylene group. Preferably such fused groups contain from 1 to 3 fused ring structures.

The term “alkoxy” refers to the groups alkyl-O—, alkenyl-O—, cycloalkyl-O— and cycloalkenyl-O—, where alkyl, alkenyl, cycloalkyl, and cycloalkenyl are as defined herein. Preferred alkoxy groups are alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups substituted alkyl-O—, substituted alkenyl-O—, substituted cycloalkyl-O— and substituted cycloalkenyl-O—, where substituted alkyl, substituted alkenyl, substituted cycloalkyl, and substituted cycloalkenyl are as defined herein. A preferred class of substituted alkoxy are polyoxyalkylene groups represented by the formula —O(R′O)_{q}R″ where R′ is an alkylene group or a substituted alkylene group, R″ is selected from the group consisting of hydrogen, alkyl or substituted alkyl and q is an integer from 1 to 10. Preferably, in such groups, q is from 1 to 5 and most preferably 3.

The term “alkenyl” refers to a monoradical of a branched or unbranched unsaturated hydrocarbon group preferably having from 2 to 40 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), n-propenyl (—CH₂CH═CH₂), iso-propenyl (—C(CH₃)═CH₂), and the like.

The term “substituted alkenyl” refers to an alkenyl group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “acyl” refers to the groups HC(O)—, alkyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)— where alkyl, substituted alkyl, cycloalkyl, substituted cycloalkyl, cycloalkenyl, substituted cycloalkenyl, aryl, heteroaryl and heterocyclic are as defined herein.

The term “acylamino” refers to the group —C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” refers to the group —NRC(O)OR where each R is independently hydrogen, alkyl, substituted alkyl, aryl, heteroaryl, or heterocyclic wherein alkyl, substituted alkyl, aryl, heteroaryl and heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Preferred aryls include phenyl, naphthyl and the like.

Unless otherwise constrained by the definition for the aryl substituent, such aryl groups can optionally be substituted with from 1 to 5 substituents, preferably 1 to 3 substituents, selected from the group consisting of acyloxy, hydroxy, thiol, acyl, alkyl, alkoxy, alkenyl, cycloalkyl, cycloalkenyl, substituted alkyl, substituted alkoxy, substituted alkenyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy.

The term “aryloxy” refers to the group aryl-O— wherein the aryl group is as defined above including optionally substituted aryl groups as also defined above.

The term “amino” refers to the group —NH₂.

The term “substituted amino refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, aryl, heteroaryl and heterocyclic provided that both R's are not hydrogen.

The term “carboxyalkyl” refers to the groups “—C(O)O-alkyl”, “—C(O)O-substituted alkyl”, “—C(O)O-cycloalkyl”, “—C(O)O-substituted cycloalkyl”, “—C(O)O-alkenyl”, and “—C(O)O-substituted alkenyl”, where alkyl, substituted alkyl, cycloalkyl, substituted cycloalkyl, alkenyl, and substituted alkenyl, are as defined herein.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl and the like.

The term “substituted cycloalkenyl” refers to cycloalkenyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “halo” or “halogen” refers to fluoro, chloro, bromo and iodo.

The term “heteroaryl” refers to an aromatic group of from 1 to 15 carbon atoms and 1 to 4 heteroatoms selected from oxygen, nitrogen and sulfur within at least one ring (if there is more than one ring).

Unless otherwise constrained by the definition for the heteroaryl substituent, such heteroaryl groups can be optionally substituted with 1 to 5 substituents, preferably 1 to 3 substituents, selected from the group consisting of acyloxy, hydroxy, thiol, acyl, alkyl, alkoxy, alkenyl, cycloalkyl, cycloalkenyl, substituted alkyl, substituted alkoxy, substituted alkenyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “heteroaryloxy” refers to the group heteroaryl-O—.

The term “heterocycle” or “heterocyclic” refers to a monoradical saturated unsaturated group having a single ring or multiple condensed rings, from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Such heterocyclic groups can have a single ring or multiple condensed rings. Preferred heterocyclics include morpholino, piperidinyl, and the like.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

The term “heterocyclooxy” refers to the group heterocyclic-O—.

The term “thioheterocyclooxy” refers to the group heterocyclic-S—.

The term “oxyacylamino” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “thiol” refers to the group —SH.

The term “thioalkoxy” refers to the group —S-alkyl.

The term “substituted thioalkoxy” refers to the group —S-substituted alkyl.

The term “thioaryloxy” refers to the group aryl-S— wherein the aryl group is as defined above including optionally substituted aryl groups also defined above.

The term “thioheteroaryloxy” refers to the group heteroaryl-S— wherein the heteroaryl group is as defined above including optionally substituted aryl groups as also defined above.

The term “poly(oxyalkylene)” refers to the group —O(R′O)_{b}R″ where R′ is an alkylene group or a substituted alkylene group, R′ is selected from the group consisting of hydrogen, alkyl or substituted alkyl, and b is an integer ranging from 1 to 10, preferably 1 to 6. One preferred poly(oxyalkylene) has the formula —O—(CH₂—CH₂—O)₃—CH₃.

The term “saccharide” refers to oxidized, reduced or substituted saccharides hexoses such as D-glucose, D-mannose, D-xylose, D-galactose, D-glucuronic acid, N-acetyl-D-glucosamine, N-acetyl-D-galactosamine, sialyic acid, iduronic acid, L-fucose, and the like; pentoses such as D-ribose or D-arabinose; ketoses such as D-ribulose or D-fructose; disaccharides such as sucrose, lactose, or maltose; derivatives such as acetals, amines, acylated, sulfated and phosphorylated sugars; oligosaccharides having from 2 to 10 saccharide units. For the purposes of this definition, these saccharides are referenced using conventional three letter nomenclature and the saccharides can be either in their open or preferably in their pyranose form.

As to any of the above groups that contain one or more substituents, it is understood, of course, that such groups do not contain any substitution or substitution patterns which are sterically impractical and/or synthetically non-feasible. In addition, the compounds of this invention include all stereochemical isomers and mixtures thereof arising from the substitution of these compounds.

The term “pharmaceutically acceptable salt” refers to salts which retain the biological effectiveness and properties of the compounds of this invention and which are not biologically or otherwise undesirable. In some cases, the compounds of this invention are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

“Protecting group” or “blocking group” refers to any group which when bound to one or more hydroxyl, thiol, amino or carboxyl groups of the compounds (including intermediates thereof) prevents reactions from occurring at these groups and which protecting group can be removed by conventional chemical or enzymatic steps to reestablish the hydroxyl, thiol, amino or carboxyl group. The particular removable blocking group employed is not critical and preferred removable hydroxyl blocking groups include conventional substituents such as allyl, benzyl, acetyl, chloroacetyl, thiobenzyl, benzylidine, phenacyl, t-butyl-diphenylsilyl and any other group that can be introduced chemically onto a hydroxyl functionality and later selectively removed either by chemical or enzymatic methods in mild conditions compatible with the nature of the product.

“Capable of localizing in the cells of a tumor or atheroma” means that following administration a moiety or compound preferentially accumulates in tumor or atheroma cells relative to any accumulation in the cells of surrounding normal tissues.

“Catalyzing moiety” refers to a moiety capable of catalyzing the production of reactive oxygen species from a cellular metabolite.

The term “cellular metabolite” or “reducing metabolite” refers to a compound found naturally within a living cell. The cellular metabolites that generate reactive oxygen species when acted upon by the compounds disclosed herein have a standard biochemical reduction potential more negative than the standard biochemical reduction potential of oxygen/hydrogen peroxide. Such metabolites include, by way of example only, NAD(P)H (i.e., NADPH and/or NADH), FADH₂, ascorbate, reduced glutathione, dihydrolipoic acid and the like.

The term “standard biochemical reduction potential” refers to the reduction potential of a metabolite measured at pH 7 and 25 C in an aqueous solution. At these conditions, oxygen and hydrogen peroxide have a reduction potential of approximately 0.273 mV. See, for example, Stryer, L. Biochem. 3^{rd} Ed. W. H. Freeman & Co., New York (1988) and The Handbook of Chemistry and Physics, CRC Press, Cleveland, Ohio. The production of reactive oxygen species, such as hydrogen peroxide, can be measured using conventional procedures, such as by measuring the disappearance of oxygen or a reducing agent, such as ascorbate, or by calorimetric assays well known in the art. For example, intracellular production of hydrogen peroxide can be monitored using dichlorofluoresein. See, for example, “Detection of Picomole Levels of Hydroperoxides Using a Florescent Dichlorofluorescein Assay”. Cathcart, R.; Schwiers, E.; Ames. B. N.; Anal. Biochem. 1983, 134, 111-116; “A Microplate Assay for the Detection of Oxidative Products using 2′,7′-Dichlorofluorescindiacetate”. Rosenkranz, A. R.; Schmaldienst, S.; Stuhimeier, K, M.; Chen, W.; Knapp, W.; Zlabinger, G. J, J. Immunol. Methods 1992, 156, 39-45; and “Generation of Reactive Oxygen Species by Human Mesothelioma Cells”. Lahlos, K.; Pitkanen, S.; Linnainmaa, K., Kinnula, V. L. Br. J. Cancer 1999, 80, 25-31.

“Conjugate” refers to two or more compounds or moieties covalently bound together.

The term “redox cycling agents” refers to compounds which may exist in two or more oxidation states, are able to lower the activation barrier for electron transfer between two compounds, and may therefore be suitable moieties for generating reactive oxygen species. Examples of suitable redox cycling agents include, for instance, alloxan, phenazine methosulfate, menadione, copper/putrescine/pyridine, methylene blue, paraquat, doxorubicin, bleomycin, and ruthenium (II) tris-(1,10-phenanthroline-5,6-dione).

“FAD” refers to the oxidized form of flavin adenine dinucleotide; and “FADH₂” refers to the reduced form of flavin adenine dinucleotide.

The term “ionizing radiation” refers to radiation conventionally employed in the treatment of tumors which radiation, either as a large single dosage or as repeated smaller dosages, will initiate ionization of water thereby forming reactive oxygen species. Ionizing radiation includes, by way of example, x-rays, electron beams, γ-rays, and the like.

“Localizing moiety” refers to a moiety capable of localizing in the cells or tissue of a tumor or atheroma or other neoplasia. Suitable localizing moieties include, by way of example, metallotexaphyrins, metalloporphyrins, monoclonal antibodies, polypeptides and like.

“NADH” refers to the reduced form of nicotinamide adenine dinucleotide; and “NAD⁺” refers to the oxidized form of nicotinamide adenine dinucleotide.

“NADPH” refers to the reduced form of nicotinamide adenine dinucleotide phosphate; and “NADP⁺” refers to the oxidized form of nicotinamide adenine dinucleotide phosphate.

The term “effective amount” means a dosage sufficient to provide treatment for the disease state being treated. This will vary depending on the patient, the disease and the treatment being effected.

“Treatment” or “treating” refers to any treatment of a pathologic condition in a mammal, particularly a human, and includes:

(i) preventing the pathologic condition from occurring in a subject which may be predisposed to the condition but has not yet been diagnosed with the condition and, accordingly, the treatment constitutes prophylactic treatment for the disease condition;

(ii) inhibiting the pathologic condition, i.e., arresting its development;

(iii) relieving the pathologic condition, i.e., causing regression of the pathologic condition; or

(iv) relieving the conditions mediated by the pathologic condition.

Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and G. M. Wuts, Protecting Groups in Organic Synthesis, Second Edition, Wiley, New York, 1991, and references cited therein.

In one embodiment, the compounds of this invention comprise a conjugate of (a) a moiety capable of localizing in the cells of a tumor or atheroma and (b) one or more moieties capable of catalyzing the production of reactive oxygen species from cellular metabolites. Any moiety capable of localizing in the cells of a tumor or atheroma may be used in this invention. Suitable localizing moieties include, by way of example, metallotexaphyrins, porphyrins, metalloporphyrins, antibodies, low density lipoproteins, saccharides, lipophilic hydrocarbyl moieties capable of association with a liposome, and the like. Such moieties are well-known in the art including derivatives thereof having functional groups suitable for use in covalently coupling a moiety capable of catalyzing the production of reactive oxygen species from a cellular metabolite to the localizing moiety. Such functional groups include, by way of example, amino, hydroxyl, thio, halo, sulfonyl and carboxyl groups and the like.

For example, polypeptides which localize in tumor tissue are described in U.S. Pat. No. 5,762,909, the disclosure of which is incorporated herein by reference in its entirety. Additionally, monoclonal antibodies which localize in tumor tissue are described, for example, in U.S. Pat. Nos. 5,965,132; 5,928,641; 5,911,969; and 5,889,157, the disclosures of which are incorporated herein by reference in their entirety.

Porphyrin derivatives and, in particular, iron(III) porphyrin may be used as the localizing moiety. Such derivatives are known to accumulate in tumor tissue and iron(III) porphyrin has been disclosed as generating hydrogen peroxide from ascorbate and oxygen. See, for example, Lin, et al., Analytical Biochemistry, The Cytotoxic Activity of Hematoheme: Evidence for Two Different Mechanisms, 161 :323-331 (1987).

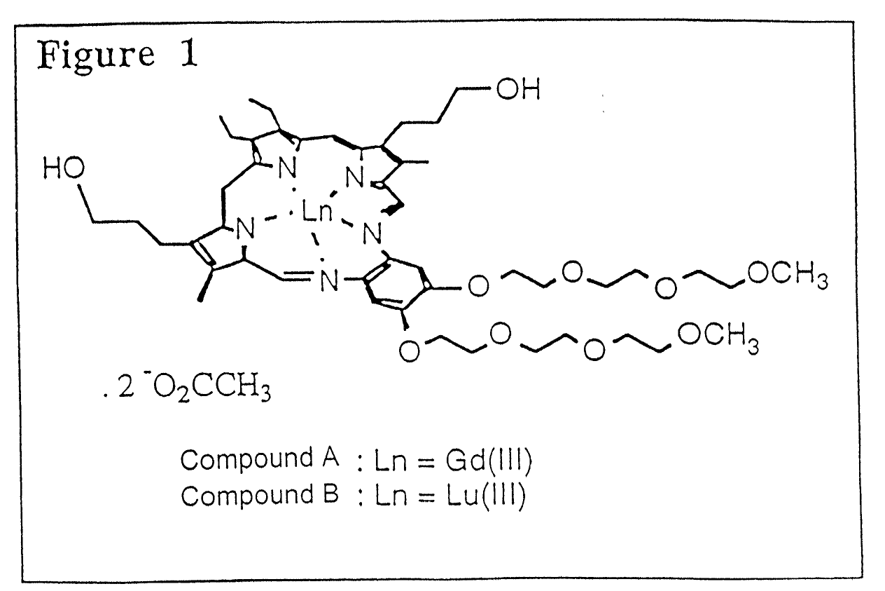

Texaphyrin compounds may be employed as the localizing moiety in this invention. Texaphyrin compounds and methods for their preparation are described in U.S. Pat. Nos. 4,935,498; 5,162,509; 5,252,720; 5,272,142, 5,256,399, 5,457,183, 5,567,687, 5,583,220 and 5,599,923; and in PCT Publication No. WO 95/21845, the disclosures of which are incorporated herein by reference in their entirety. Texaphyrin refers to an “expanded porphyrin” pentadentate macrocyclic ligand as shown by way of example in FIG. 1. Such compounds are capable of existing in both a free-base form and in a 1:1 complex form with a variety of metal cations, including divalent metal cations such as Ca(II), Cd(II), Mn(II), Co(II), Ni(II), Zn(II), Hg(II), Fe(II), Sm(II) and UO₂(II); and trivalent metal cations such as Mn(III), Co(III), Ni(III), Fe(III), Ho(III), Ce(III), Y(III), ln(lli), Pr(III), Nd(III), Sm(III), Eu(III), Gd(III), Tb(III), Dy(III), Er(III), Tm(III), Yb(III), Lu(III), La(III) and U(III); and other cations.

Particularly preferred texaphyrins include those represented by formula III:

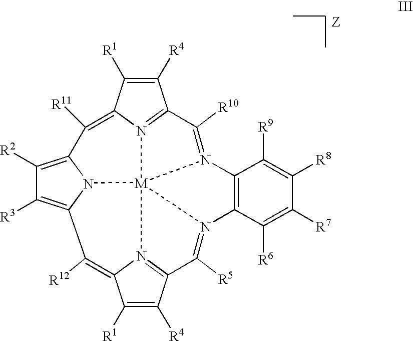

wherein:

M is a divalent metal cation or a trivalent metal cation;

R¹ to R⁴ as well as R⁷ and R⁸ are independently selected from the group consisting of hydrogen, carboxyl, carboxylalkyl, acyl, acylamino, aminoacyl, alkyl, substituted alky (particularly hydroxyalkyl or aminoalkyl, and especially where R¹ is hydroxypropyl or aminopropyl), alkenyl, substituted alkenyl, alkoxy, substituted alkoxy, aryl, heteroaryl, heterocyclic, halo, hydroxyl, nitro, and a saccharide;

R⁶ and R⁹ are independently selected from the group consisting of hydrogen, carboxyl, carboxylalkyl, acyl, acylamino, aminoacyl, alkyl, substituted alkyl other than iodoalkyl, alkenyl, substituted alkenyl, alkoxy, substituted alkoxy, aryl, heteroaryl, heterocyclic, halo other than iodo, hydroxyl, nitro, and a saccharide;

R⁵ and R¹⁰ to R¹² are independently selected from the group consisting of hydrogen, alkyl, substituted alkyl, alkoxy, substituted alkoxy, carboxyl, carboxylalkyl, acyl and acylamino; and

the charge, Z, is an integer having a value less than or equal to 5.

The divalent or trivalent metal M is preferably selected from the group consisting of Ca(II), Mn(II), Co(II), Ni(II), Zn(II), Cd(II), Hg(II), Fe(II), Sm(II), UO₂(II), Mn(Ill), Co(III), Ni(III), Fe(III), Ho(III), Ce(III), Y(III), In(III), Pr(III), Nd(III), Sm(III), Eu(III), Gd(III), Tb(III), Dy(III), Er(III), Tm(III), Yb(III), Lu(III), La(III), and U(III).

Particularly preferred texaphyrin compounds are represented by formula IV:

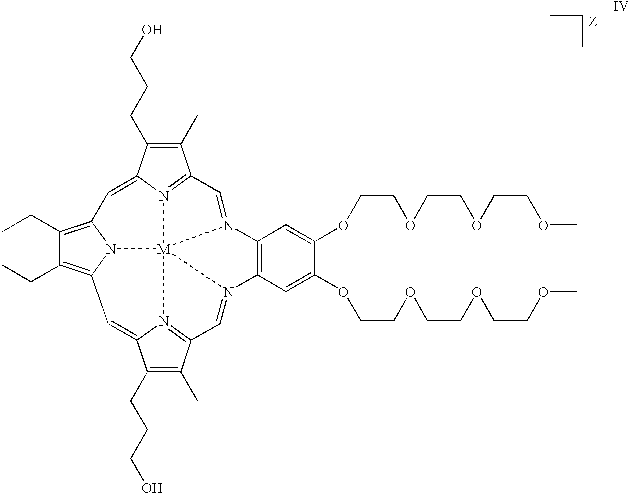

wherein M and Z are as defined above. In compounds of formula IV, the hydroxyl groups serve as useful functional groups for covalently attaching linkers and/or catalyzing moieties to the metallotexaphyrin.

Preferably, in formula IV, M is Gd(III) and Z is +2; M is Dy(III) and Z is +2; M is Y(III) and Z is +2; M is Lu(III) and Z is +2; M is Co(II) and Z is +1; and M is Mn(II) and Z is +1. Most preferably, M is Gd(III) or Lu(III).

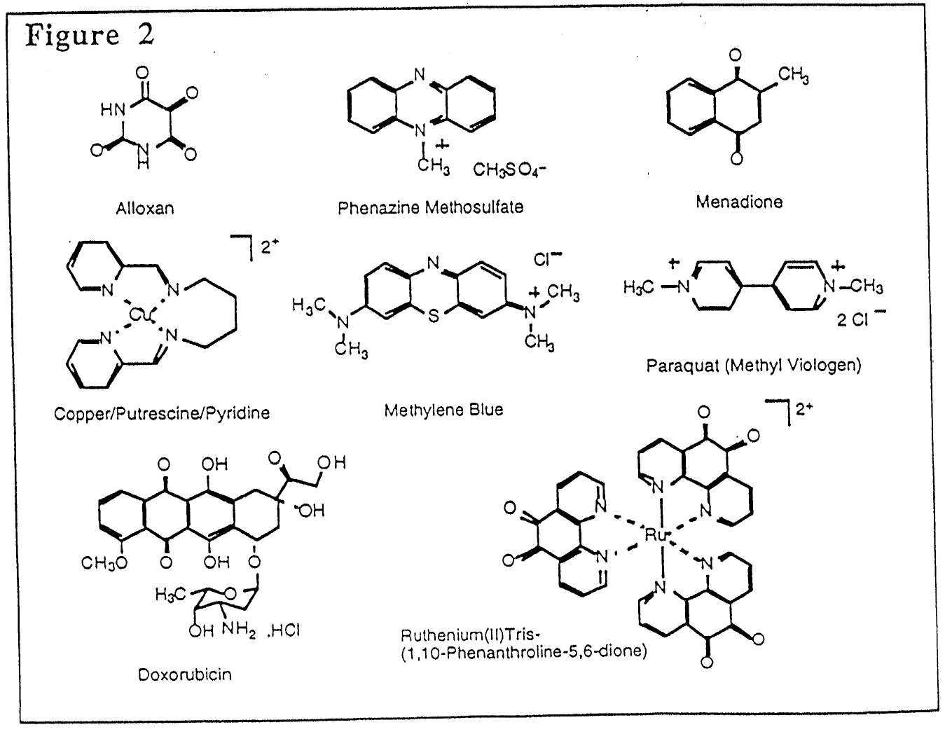

The localizing moiety employed in this invention is typically coupled to one or more moieties capable of catalyzing the production of reactive oxygen species from cellular metabolites. Any moiety capable of catalyzing the production of reactive oxygen species from a cellular metabolite having a standard biochemical reduction potential more negative than the standard biochemical reduction potential of oxygen/hydrogen peroxide, i.e, about 0.273 mV, may be used in this invention. Suitable catalyzing moieties include, by way of illustration, alloxan, phenazonium salts, quinones such as menadione, copper/putrescine/pyridine, methylene blue, paraquat (methyl viologen), doxorubicin and the like, and derivatives and salts thereof. The structures of these materials are shown in FIG. 2. The reduction potential of these compounds can be altered by substitution with one or more electron withdrawing groups, such as halo, nitro, formyl and the like. Additionally, various redox mediators are discussed in “Oxidative Stress Induced by a Di-Schiff Base Copper Complex is both mediated and Modulated by Glutathione.” Steinkuhler, C.; Pedersen, J. Z.; Weser, U.; Rotilio. G. Biochem. Pharmacol. 1991, 42, 1821-1827; “Stimulation of respiration by Methylene Blue in Rat Liver Mitochondria”. Visarius, T. M.; Stucki, J. W.; Lauterburg, B. H. FEBS Left. 1997, 412, 157-160; “Unimpaired Metabolism of Pyridine Dinucleotides and Adenylates in Chinese Hamster Ovary Cells During Oxidative Stress Elicited by Cytotoxic Doses of Copper-Putrescine-Pyridine”. Nagele, A. Biochem. Pharmacol. 1995, 49, 147-155; “Protein-Ubiquinone Interaction in Bovine Heart Mitochondrial Succinate-Cytochrome c Reductase”. Yang, F.; Yu, L.; He, D.; Yu, C. J. Biol. Chem. 1991, 266. 20863-20869; “Interaction of the Antitumor Drug Streptonigrin with Palladium(II) Ions. Evidence of the Formation of a Superoxo-Palladium(II)-Streptonigrin Complex”. Fiallo, M. M. L.; Gamier-Suillerot, A. Inorg. Chem. 1990, 29, 893-897; “Efficient Catalyic Systems for Electron Transfer from a NADH Model Compound to Dioxygen”. Inorg. Chem. 1990, 29, 653-659; “Preparation and Kinetic Properties of 5-Ethylphenazine-Poly(Ethylene Glycol)-NAD+ Conjugate, a Unique Catalyst Having an Intramolecular Reaction Step”. Yomo, T.; Sawai, H.; Urabe, I.; Okada H. Eur. J. Biochem. 1989, 179, 299-305; “Mediator Compounds for the Electrochemical Study of Biological Redox Systems: A Compilation”. Fultz, M. L.; Durst, R. A. Analyt. Chim. Acta. 1982, 140,1-18; “An Electrochemical Study of the Kinetics of NADH Being Oxidized by Diimines Derived from Diaminobenzenes and Diaminopyrimidines”. Kitani, A.; So, Y. —H.; Miller, L. L. J. Am. Chem. Soc.1981, 103, 7636-7641; “Fast Oxidants for NADH and Electrochemical Discrimination between Ascorbic Acid and NADH” Kitani, A.; Miller, L. L.; J. Am. Chem. Soc. 1981, 103, 3395-3397; and “Oxidation of Dihydronicotinamide Adenine Dinucleotide by Flavin Derivative of Polythyleneimine” Spetnagel, W. J.; Klotz, I. M. Biopolymers 1978, 17, 1657-1668.

The compounds of this invention typically contain from 1 to about 5 catalyzing moieties, preferably 1 or 2 catalyzing moieties, per localizing moiety. When two or more catalyzing moieties are employed, the catalyzing moieties may be the same or different. If desired, the catalyzing moiety may be derivatized to improve the solubility and/or biodistribution of the conjugate, i.e., by substitution with one or more poly(oxyalkylene) groups.

Coordination of nitroimidazole with porphyrin has been suggested as a potential targeting approach (Brunner, H. et al., Chem. Ber., 1995, 128:173-181; Chem. Ber. 1994, 127:2141-2149). To the extent that nitroimidazole may subsequently be found to catalyze the production of reactive oxygen species from a cellular metabolite and thereby inherently fall within the mechanism disclosed in in co-pending U.S. patent application Ser. No. ______, filed on even date herewith and entitled “Methods and Compositions for Treating Atheroma, Tumors and Other Neoplastic Tissue” (Attorney Docket No. 032790-002), it is intended that such conjugates be excluded from the scope of the present claims. Similarly, to the extent that texaphyrins or other localizing agents have been conjugated to moieties subsequently found to catalyze the production of reactive oxygen species from a cellular metabolite, it is intended that such conjugates also be excluded from the scope of the present claims.

Generally, the catalyzing moiety(s) are covalently bound to the localizing moiety with a linking group. Any linking group which covalently attaches the catalyzing moiety(s) to the localizing moiety may used in the compounds of this invention, including a covalent bond.

The linking group can be represented by formula V:

—X^{a}-Z-(Y^{a}-Z)ₚ—Y^{b}-Z-X^{a}— V

in which:

p is an integer of from 0 to 20;

X^{a} at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;

Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;

Y^{a} and Y^{b} at each separate occurrence are selected from the group consisting of:

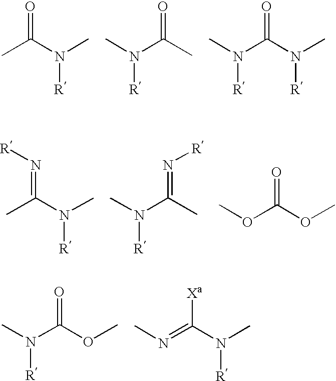

—S(O)_{q}—CR′R″—, —S(O)_{q}—NR′—, —S—S—, or a covalent bond;

in which:

q is 0, 1 or 2; and

R, R and R at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

To the extent that a covalent bond is selected for adjacent substituents in the linkers of formula V (e.g., X^{a} and Z are adjacent and can each be a covalent bond) the adjacent substituents should be understood to comprise a single covalent bond.

Additionally, the linker moiety can be optionally substituted at any atom therein by one or more alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic group.

Particularly preferred linking groups are alkylene groups having from 1 to 20 carbon atoms; poly(oxyalkylene) groups having from 2 to 20 carbon atoms and from 1 to 10 oxygen atoms; and a covalent bond. A preferred alkylene linking group has the formula: —(CH₂)ₙ—, where n is an integer ranging from 1 to about 20, preferably from 2 to 6.

The catalyzing moiety(s) may be covalently attached to the localizing moiety via the linking group using conventional coupling procedures well known in the art. Reaction chemistries for forming covalent linkages are well known in the art and involve the use of complementary functional groups on the moieties to be coupled together. Preferably, the complementary functional groups on each moiety are selected relative to the functional groups available on the other moiety for bonding or which can be introduced onto the other moiety for bonding. Again, such complementary functional groups are well known in the art. For example, reaction between a carboxylic acid on one moiety and a primary or secondary amine of the other moiety in the presence of suitable, well-known activating moieties results in formation of an amide bond covalently linking the two moieties; reaction between an amine group of one moiety and a sulfonyl halide of the other moiety results in formation of a sulfonamide bond covalently linking the two moiety; and reaction between an alcohol or phenol group of one moiety and and an alkyl or aryl halide of the other moiety results in formation of an ether bond covalently linking the two moiety. A wide variety of other complementary chemistries are well known to those skilled in the art. By way of example, the Mitsunobu reaction is particularly useful for coupling texaphyrin and porphyrin derivatives to various catalyzing moieties. The Mitsunobu reaction is further described in Mitsunobu, O., Synthesis, 1981, 1-28.

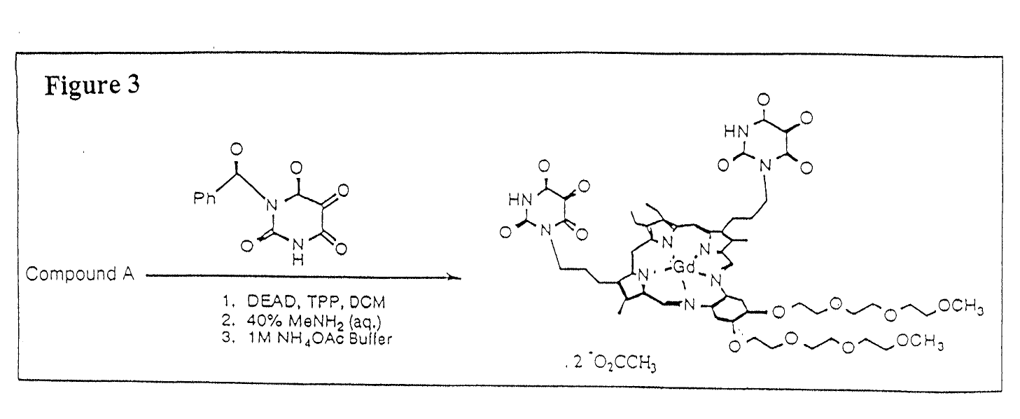

Similarly, FIG. 3 illustrates the coupling of a protected alloxan deriviative to the bis-hydroxy metallotexaphyrin using the Mitsunobu reaction. In this reaction, 3-benzoylalloxan is first coupled to the bis-hydroxy metallotexaphyrin under Mitsunobu reaction conditions as described above. The benzoyl protecting groups are then removed using standard deprotection conditions, i.e., treatment with 40% aqueous methylamine at ambient temperature, to afford the coupled product shown in FIG. 3.

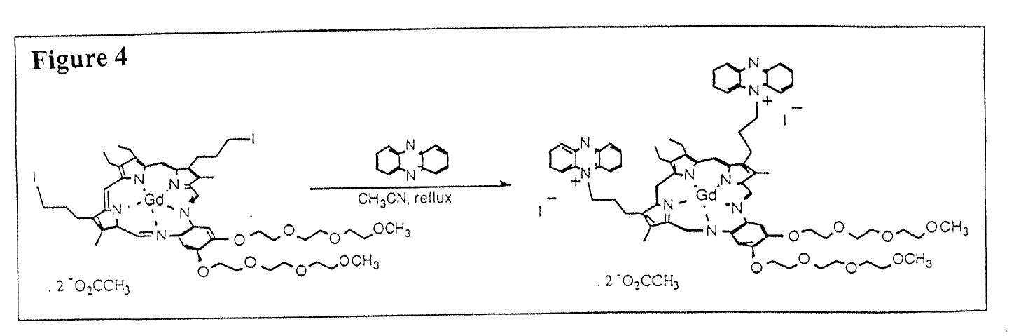

Alternatively, the bis-iodo metallotexaphyrin shown in FIG. 4, (prepared by a conventional modification of the Mitsunobu reaction) can be employed to couple a catalyzing moiety to the localizing moiety. As shown in FIG. 4, the bis-iodo metallotexaphyrin can be reacted with 2.0 to about 2.3 equivalents of phenazine in refluxing acetonitrile to afford the coupled product shown in FIG. 4. The bis-iodo metallotexaphyrin shown in FIG. 4 is a useful synthon for preparing other compounds of this invention via displacement of the iodo groups.

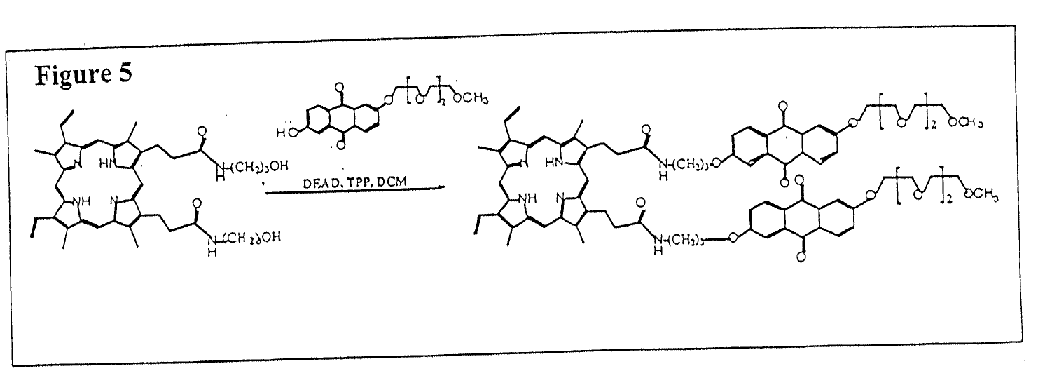

Finally, the coupling of a quinone deriviative to a bis-hydroxy porphyrin is illustrated in FIG. 5. In this reaction, two equivalents of the quinone are coupled to the bis-hydroxy porphyrin using the Mitsunobu reaction as described above. The quinone employed in this reaction is derivatized with two polyoxyethylene groups to increase the aqueous solubility of the coupled product.

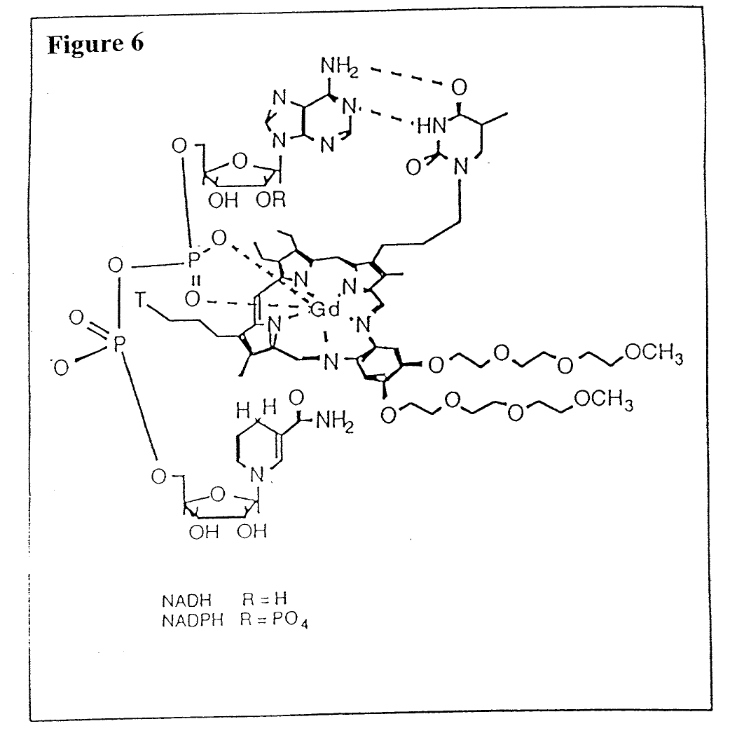

The compounds of formula II can be prepared using procedures similar to those described herein for the compounds of formula I. For the compounds of formula II, a localizing moiety capable of catalyzing the production of reactive oxygen species from a cellular metabolite having a standard biochemical reduction potential more negative than the standard biochemical reduction potential of oxygen/hydrogen peroxide is chosen, such as a gadolinium texaphyrin, and one or more ligands capable of binding to NADH or NADPH are covalently attached thereto using conventional reagents and procedures. Any ligand capable of binding to NADH or NADPH may be employed including, by way of illustration, thymine and thymine derivatives. The binding of a compound of formula II to NADH or NADPH is illustrated in FIG. 6.

The preferred compounds of the present invention include the following:

Complexes of a metallotexaphyrin linked to a thymidine base.

Complexes of a porphyrin or metalloporphyrin, particularly protoporphyrin 9, linked to a redox cycling agent capable of generating reactive oxygen species from a cellular metabolite having a standard biochemical reduction potential more negative than the standard biochemical reduction of oxygen/hydrogen, such as alloxan, phenazonium salts, a quinone and deriviatives and/or salts thereof, preferably hydroxyanthraquinone. Particularly preferred are those complexes linked by a covalent bond, an alkylene group and a poly(oxyalkylene) group, optionally including an amidocarboxy or carboxamide functionality

Complexes of an antibody and a redox cycling agent.

Pharmaceutical Formulations

When employed as pharmaceuticals, compounds described herein are usually administered in the form of pharmaceutical compositions. These compounds can be administered by a variety of routes including oral, intravenous, intramuscular, and the like. These compounds are effective as both injectable and oral compositions. Such compositions are prepared in a manner well known in the pharmaceutical art and comprise at least one active compound.

These pharmaceutical compositions contain, as the active ingredient, one or more of the compounds described herein associated with pharmaceutically acceptable carriers. In making these compositions, the active ingredient is usually mixed with an excipient. When the excipient serves as a diluent, it can be a solid, semi-solid, or liquid material, which acts as a vehicle, carrier or medium for the active ingredient. Thus, the compositions can be in the form of tablets, pills, elixirs, suspensions, emulsions, solutions, syrups, and the like containing, for example, up to 10% by weight of the active compound, soft and hard gelatin capsules, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of formula I above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The following formulation examples illustrate representative pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50 to 60 C and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### FORMULATION EXAMPLE 4

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, starch, and magnesium stearate are blended, passed through a No. 20 mesh U.S. sieve, and filled into hard gelatin capsules in 150 mg quantities.

### FORMULATION EXAMPLE 5

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 6

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, starch, and magnesium stearate are blended, passed through a No. 20 mesh U.S. sieve, and filled into hard gelatin capsules in 425.0 mg quantities.

### FORMULATION EXAMPLE 7

An intravenous formulation may be prepared as follows:

| Ingredient | Quantity | |
|---|---|---|
| Active Ingredient | 250.0 | mg |
| Mannitol | 50.0 | mg |
| Water (distilled, sterile) | qs. to 1000 | mL |

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents. Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference. Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Other suitable formulations for use in the present invention can be found in Remington's Pharmaceutical Sciences, Mace Publishing Company, Philadelphia, Pa., 17th ed. (1985).

Utility

The compounds of this invention localize in tumor or atheroma cells and catalyze the in situ production of reactive oxygen species from cellular metabolites. Accordingly, when administered to a mammalian host having a tumor or atheroma, the compounds of this invention selectively catalyze the production of reactive oxygen species in the tumor or atheroma cells thereby killing or treating the tumor or atheroma. Accordingly, the compound of this invention are useful for treating atheroma, tumors and other neoplastic tissues.

The amount of compound administered to the patient will vary depending upon what compound and/or composition is being administered, the purpose of the administration, such as prophylaxis or therapy, the state of the patient, the manner of administration, and the like. In therapeutic applications, compositions are administered to a patient already suffering from, for example, atheroma or a tumor in an amount sufficient to at least partially reduce the growth of the atheroma or tumor. Amounts effective for this use will depend on the judgment of the attending clinician depending upon factors such as the type of atheroma or tumor in the patient and its size, the age, weight and general condition of the patient, and the like. The pharmaceutical compositions of this invention may contain more than one compound of the present invention or other active drugs or materials.

As discussed above, the compounds described herein are suitable for use in a variety of drug delivery systems. The compositions can be administered by different routes including intravenously, intraperitoneally, subcutaneously, intramuscularly, orally, topically, or transmucosally.

The amount of compound administered to the patient will vary depending upon what is being administered, the purpose of the administration, the state of the patient, the manner of administration, and the like. In particular, a sufficient amount of the compound is administered to the cell or to the patient so as to generate reactive oxygen species in quantities effective to initiate tumor cell death. An amount adequate to accomplish this is defined as “therapeutically effective dose.” Amounts effective for this use will depend on the judgment of the attending clinician depending upon factors such as the degree or severity of the cancer in the patient, the age, weight and general condition of the patient, and the like. Preferably, when so employed, the compound is administered at dosages ranging from about 0.1 to about 100 mg/kg/day.

The compounds of this invention can also be administered in conjunction with radiation treatment. When employed with ionizing radiation, the amount of compound administered to the patient will vary depending upon what is being administered, the purpose of the administration, the state of the patient, the manner of administration, and the like. In particular, a sufficient amount of the compound is administered to the cell or to the patient to therapeutically enhance the effect of ionizing radiation on tumor cell death. An amount adequate to accomplish this is defined as “therapeutically effective dose.” Amounts effective for this use will depend on the judgment of the attending clinician depending upon factors such as the degree or severity of the cancer in the patient, the age, weight and general condition of the patient, and the like. Preferably, compounds used in conjunction with ionizing radiation are administered at dosages o ranging from about 0.1 to about 100 mg/kg/day.

If desired, the composition can be administered at short time intervals using a pump to control the time interval or achieve continuously administration. Suitable pumps are commercially available (e.g., the ALZET® pump sold by Alza corporation, and the BARD ambulatory PCA pump sold by Bard MedSystems).

Plasma half-life and biodistribution of the drug and metabolites in the plasma, tumors, and major organs can be also be determined to facilitate the selection of drugs most appropriate to inhibit a disorder. Such measurements can be carried out, for example, using HPLC analysis from dissected animals treated with the drug. Compounds that show potent activity in the screening assays, but have poor pharmacokinetic characteristics, can be optimized by altering the chemical structure and retesting. In this regard, compounds displaying good pharmacokinetic characteristics can be used as a model.

As noted above, the compounds administered to a patient are in the form of pharmaceutical compositions described herein. These compositions may be sterilized by conventional sterilization techniques, or may be sterile filtered. When aqueous solutions are employed, these may be packaged for use as is, or lyophilized, the lyophilized preparation being combined with a sterile aqueous carrier prior to administration. The pH of the compound preparations typically will be between 3 and 11, more preferably from 5-9 and most preferably from 7 and 8. It will be understood that use of certain of the foregoing excipients, carriers, or stabilizers will result in the formation of pharmaceutical salts.

By way of example for the methods of the invention further employing the administration of ionizing radiation, the radiation sensitizer motexafin gadolinium is administered in a solution containing 2 mM optionally in 5% mannitol USP/water (sterile and non-pyrogenic solution). Dosages of 0.1 mg/kg up to as high as about 23.0 mg/kg have been delivered, preferably about 3.0 to about 15.0 mg/kg (for volume of about 90 to 450 mL) may be employed, optionally with pre-medication using anti-emetics above about 6.0 mg/kg. The texaphyrin is administered via intravenous injection over about a 5 to 10 minute period, followed by a waiting period of about 2 to 5 hours to facilitate intracellular uptake and clearance from the plasma and extracellular matrix prior to the administration of radiation.

When employing radiation therapy, a palliative course of 30 Gy in ten (10) fractions of radiation are typically administered over consecutive days excluding weekends and holidays. In the treatment of brain metastases, whole brain megavolt radiation therapy is delivered with 60Co teletherapy or a >4 MV linear accelerator with isocenter distances of at least 80 cm, using isocentric techniques, opposed lateral fields and exclusion of the eyes. A minimum dose rate at the midplane in the brain on the central axis is about 0.5 Gy/minute.

Radiation sensitizers may be administered before, or at the same time as, or after administration of the ionizing radiation, preferably before. The radiation sensitizer may be administered as a single dose, as an infusion, or it may be administered as two or more doses separated by an interval of time. Where the radiation sensitizer is administered as two or more doses, the time interval between administrations may be from about one minute to a number of days, preferably from about 5 min to about 1 day, more preferably about 4 to 5 hr. The dosing protocol may be repeated, from one to ten or more times, for example. Dose levels for radiation sensitization using motexafin gadolinium may range from about 0.05 μmol/kg to about 20 μmol/kg administered in single or multiple doses (e.g. before each fraction of radiation). A lower dosage range is presently preferred for intra-arterial injection or for impregnated stents. In the case of texaphyrins incorporating or conjugated to a radioisotope, the additional administration of radiation as a co-therapeutic agent is optional.

Administering a radiation sensitizer to a mammalian host bearing atheroma cells may be prior to, concurrent with, or following vascular intervention, and the intervention is followed by radiation. The administration may begin prior to, such as about 24-48 hours prior to, or at a time roughly accompanying vascular intervention, for example. Multiple or single treatments prior to, at the time of, or subsequent to the procedure may be used. “Roughly accompanying the vascular intervention” refers to a time period within the ambit of the effects of the vascular intervention. Typically, an initial dose of the sensitizer and radiation will be within 1-24 hours of the vascular intervention, preferably within about 5-24 hours thereafter. Follow-up dosages may be made at weekly, biweekly, or monthly intervals. Design of particular protocols depends on the individual subject, the condition of the subject, the design of dosage levels, and the judgment of the attending practitioner.

The following examples are offered to illustrate this invention and are not to be construed in any way as limiting the scope of this invention.

### EXAMPLES

### Example 1

### Synthesis of the Compound of FIG. 3

Step 1: To a stirred solution of Compound A as shown in FIG. 1 (prepared as described in U.S. Pat. No. 5,457,183) (1 mmol) in methylene chloride (10 mL) under nitrogen is added triphenylphosphine (2.2 mmol) followed by diethylazodicarboxylate (2.2 mmol) at room temperature. 3-Benzoylalloxan (2.2 mmol) is added and stirring is continued. The course of the reaction is followed by HPLC. After reaction is complete, the reaction mixture is concentrated in vacuoand the desired intermediate is obtained by purification of the crude product by use of reverse-phase column chromatography.

Step 2: A solution of the intermediate prepared in Step 1 above, in methylamine (10 mL, 40 wt. % solution in water) is stirred at room temperature. After reaction is complete, the reaction mixture is concentrated to dryness and the crude intermediate is used in the next step without further purification.

Step 3: A solution of the intermediate prepared in Step 2 above, in ammonium acetate buffer (10 ml, 1 M solution) is stirred at room temperature. After reaction is complete, the reaction mixture is concentrated to dryness and the desired product shown in FIG. 3 is obtained by purification of the crude product by use of HPLC.

### Example 2

### Synthesis of the Compound of FIG. 4

A solution of bis-iodo compound of FIG. 4 (1 mmol) and phenazine (2 mmol) in acetonitrile is heated at reflux. After the reaction is complete, the reaction mixture is concentrated to dryness and the desired shown in FIG. 4 is obtained by purification of the crude product by use of HPLC.

### Example 3

### In vivo Tumor Inhibition

In vivo tumor inhibition can be measured using a subcutaneous Xenograft model. Mice (BALB/c, nu/nu) are implanted with C6 glioma cells and the ability of compounds of this invention to inhibit tumor growth can be measured.

C6 cells are maintained in Ham's F10 supplemented with 10% fetal bovine serum (FBS) and 2 mM glutamine (GLN). Cells are harvested at or near confluence with 0.05% Trypsin-EDTA and pelleted at 450× g for 10 min. Pellets are resuspended in sterile PBS or media (without FBS) to a particular concentration and the cells are implanted into the hind-flank of mice. Tumor growth is measured over 3 to 6 weeks using venier calipers. Tumor volumes are calculated as a product of length x width x height. The test compounds are solubilized in 50-100 μL vehicle (DMSO) or dissolved in PBS (pH 7.4). The compounds are delivered by IP injection at 15 mg/kg/day. Optionally, ionizing radiation is administered after a suitable waiting period when the catalytic moiety is a radiation sensitizer. A reduction in tumor volume compared to untreated controls indicates that tumor growth is inhibited.

From the foregoing description, various modifications and changes in the compositions and methods of this invention will occur to those skilled in the art. All such modifications coming within the scope of the appended claims are intended to be included therein.

What is claimed is:

1. A compound comprising a conjugate of (a) a moiety capable of localizing in the cells of a tumor or atheroma and (b) a moiety capable of catalyzing the production of reactive oxygen species from a cellular metabolite having a standard biochemical reduction potential more negative than the standard biochemical reduction potential of oxygen/hydrogen peroxide, or a pharmaceutically acceptable salt thereof.

2. A compound of formula I:
A-[-L-X]ₙ I
wherein
A is a moiety capable of localizing in the cells of a tumor or atheroma;
each X is independently a moiety capable of catalyzing the production of reactive oxygen species from a cellular metabolite having a standard biochemical reduction potential more negative than the standard biochemical reduction potential of oxygen/hydrogen peroxide;
each L is independently a linking group covalently attaching X to A;
n is an integer ranging from 1 to 5;
and pharmaceutically acceptable salts thereof.

3. The compound of claim 2, wherein A is a metallotexaphyrin.

4. The compound of claim 2, wherein A is a porphyrin, metalloporphyrin, antibody, low density lipoprotein, saccharide, or lipophilic hydrocarbyl moiety capable of association with a liposome.

5. The compound of claim 2, wherein each X is independently selected from the group consisting of alloxan, phenazonium salts, a quinone and deriviatives and/or salts thereof.

6. The compound of claim 2, wherein each L is independently selected from the group consisting of a covalent bond, an alkylene group and a poly(oxyalkylene) group, optionally including an amidocarboxy or carboxamide functionality.

7. The compound of claim 2, wherein n is 1 or 2.

8. The compound of claim 2, wherein the cellular metabolite is selected from consisting of ascorbate, NADPH, NADH, FADH₂ and reduced glutathione.

9. A compound of formula II:
B-[-L-Y]ₙ II
wherein
B is a moiety capable of localizing in the cells of a tumor or atheroma and which is capable of catalyzing the production of reactive oxygen species from a cellular metabolite having a standard biochemical reduction potential more negative than the standard biochemical reduction potential of oxygen/hydrogen peroxide;
each Y is independently a ligand capable of binding to NADH or NADPH;
each L is independently a linking group covalently attaching Y to A; and
n is an integer ranging from 1 to 5;
and pharmaceutically acceptable salts thereof.

10. The compound of claim 8, wherein A is a metallotexaphyrin.

11. The compound of claim 8, wherein A is a porphyrin, metalloporphyrin, antibody, low density lipoprotein, saccharide, or lipophilic hydrocarbyl moiety capable of association with a liposome.

12. The compound of claim 8, wherein each Y is thymine or a derivative thereof.

13. The compound of claim 8, wherein each L is independently selected from the group consisting of a covalent bond, an alkylene group and a poly(oxyalkylene) group, optionally including an amidocarboxy or carboxamide functionality.

14. The compound of claim 8, wherein n is 1 or 2.

15. The compound of claim 8, wherein the cellular metabolite is selected from consisting of ascorbate, NADPH, NADH, FADH₂ and reduced glutathione.

16. A pharmaceutical composition comprising a pharmaceutically acceptable carrier and a therapeutically effective amount of a compound of claim 1, 2 or 9.

17. A method of treating a mammalian host having a tumor or atheroma, the method comprising:
(A) administering to a mammalian host having a tumor or atheroma an effective amount of a compound of claim 1, 2 or 9.

18. The method of claim 17, wherein the method further comprises the step of:
(B) exposing the tumor or atheroma to ionizing radiation.
